(19) United States
(12) Patent Application Publication
(10) Pub. No.: US 2013/0058922 A1
(43) Pub. Date: Mar. 7, 2013

(54) PHARMACEUTICAL COMPOSITION CONTAINING CHOLINE

(71) Applicant: Ernst-Maria App, Freiburg im Breisgau (DE)

(72) Inventor: Ernst-Maria App, Freiburg im Breisgau (DE)

(21) Appl. No.: 13/508,159

(22) PCT Filed: Nov. 3, 2010
(86) PCT No.: PCT/EP2010/066747

§ 371 Date: Nov. 19, 2012

(30) Foreign Application Priority Data
Nov. 6, 2009 (DE) 10 2009 053 157.2

(51) Int. Cl. A61K 31/14; A61P 35/00; A61K 31/7068; A61K 39/385; A61K 31/337; C07C 215/40; A61P 35/02

(52) U.S. Cl. 424/133.1; 564/293; 514/642; 562/585; 514/449; 514/49

(57) ABSTRACT

The invention relates to pharmaceutical compositions, in particular for intravenous administration, containing choline or pharmaceutically acceptable salts or analogues thereof.

The invention further relates to pharmaceutical compositions, in particular for intravenous administration, containing choline or pharmaceutically acceptable salts or analogues thereof, alone or in combination with vitamin C and/or L-arginine and/or vitamin D3 (cholecalciferol) for use in the fight against CTCs, DTCs or dysfunctional leukocyte precursors in the blood or bone marrow.

The invention relates to pharmaceutical compositions containing choline or pharmaceutically acceptable salts and analogues thereof for use in the fight against circulating tumour cells (CTCs) in the blood and disseminated tumour cells in the bone marrow (DTCs) or morphologically undifferentiated and dysfunctional leukocyte precursors in the blood and bone marrow.

The present invention relates, in particular, to pharmaceutical compositions for intravenous administration containing choline or pharmaceutically acceptable salts and analogues thereof for use in early treatment of a tumour disease of patients without manifest primary tumour, with CTCs, DTCs or morphologically undifferentiated and dysfunctional leukocyte precursors being present in the blood and bone marrow.

The invention further relates to pharmaceutical compositions, in particular for intravenous administration, containing choline or pharmaceutically acceptable salts and analogues thereof for use in tumour therapy for the reduction of metastasis risk and/or for the prophylaxis and early treatment of metastases (distant metastasis) as well as in therapy of leukaemias.

Moreover, the invention relates to pharmaceutical compositions, in particular for intravenous administration, containing choline or pharmaceutically acceptable salts and analogues thereof for use in tumour therapy in cases where, after surgical therapy, radiotherapy or chemotherapy, full remission (no detection of tumour and metastases) is diagnosed with imaging methods even though, in the blood or bone marrow, CTCs or DTCs are still present (late therapy), and in the therapy of tumour patients after, according to common criteria, unsuccessful therapy for Ultima Ratio Therapy.

The invention further relates to pharmaceutical compositions, in particular for intravenous administration, containing choline or pharmaceutically acceptable salts thereof in combination with vitamin C and/or L-arginine and/or vitamin D3 (chlolecalciferol) or pharmaceutically acceptable salts thereof.

In this context, tumour diseases according to the present invention are in particular carcinomas, sarcomas, neuroendocrine tumours, hematopoietic tumours such as leukemias and lymphomas as well as dysontogenic tumours, i.e. solid tumours as well as tumours of the hematogenic system.

### PRIOR ART

Even though choline is often referred to as vitamin B4 and, in earlier documents, also as vitamin J, it is, in fact, no essential nutrient: choline is taken in with food by monogastric mammals since choline, as a component of phosphatidylcholines, is present in the cell membrane.

With adequate supply of amino acids, choline can usually be taken up from food and produced in the body in physiologically sufficient amounts. Thus, 5 gram lecithin contain 1 gram phosphatidylcholine which is to be equivalent to the assumed minimum requirement per day. The uptake of choline in the human body can, however, be affected by the intake of certain substances such as sugar, alcohol and tea while the ingestion of folic acid, inositol and vitamin B12 complex preparations can enhance the absorption of choline. Stress may result in increased consumption which then leads to choline deficiency. Thus, wrong diet, diseases and stress result in choline deficiency which can be compensated by the administration of the pharmaceutical composition of the invention into the milieu intérieur.

As a component of phosphatidylcholine (lecithin), choline is ubiquitous in the cell membranes and, thus, it is contained in all cells of the body.

The prior art describes the advantageous effect of choline on memory and the ability to concentrate.

Choline is also said to play a role in the metabolism and the transport of triglycerides and other fats. According to this role, it is also used in oral therapy aiming at supporting liver and gallbladder function in fat utilization and reducing the cholesterol level. Due to the above-mentioned properties, choline is also used as nutritional supplement to be orally administered in form of capsules of up to 200 g.

From examinations by means of nuclear magnetic resonance spectroscopy, it is known that the concentration of choline in prostate cancer cells is significantly increased in comparison to normal cells. For this reason, choline is used in form of C-11 choline, which is administered intravenously, for the imaging diagnostic of prostate cancer.

Giambarresi et al. (Br. J. Cancer (1982) 46, 825-829) describe that, in the mouse model, a choline-free diet promotes the development of liver carcinomas.

Becker J., Zeitschrift für Krebsforschung, 1948, Vol. 56, p. 171-175, describes the use of choline as a chemotherapeutic agent for manifest malignant tumours.

WO 94/06413 discloses the use of compounds of the class of trimethylamine oxides for cancer prophylaxis.

Xu et al. (The FASEB Journal, published online on 27 Jul. 2009: High intakes of choline and betaine reduce breast cancer mortality in a population-based study) describe a study analysing the relation between the development of breast cancer and the oral dietary intake of choline and betaine. This article postulates a prophylactic effect of a high oral intake of choline and betaine via food. For the female patients [of this study], a total amount of choline from food intake was calculated which was between <196.5 mg/day and >455.8 mg/day.

Xu et al., FASEB Journal 2008; 22:2045-2052, further describe that, in a study, the breast cancer risk was inversely proportional to the choline amount taken up with food.

For a short time, it has been known that circulating tumour cells in the blood can be detected not only with clinically manifest tumour disease but already at a prior stage, before a tumour can be detected by means of imaging methods.

The detection of circulating tumour cells (CTCs) in the blood is of particular importance with respect to tumour diseases since the blood reflects a dynamic process. The survival period of 24 hours of CTCs in blood is relatively short, cf. e.g. Patel at al. 2002, Ann. Surg. 235(2): 226-31. It is unlikely that these cells will be detected incidentally, unless there is a permanent new influx of tumour cells. For this reason, the detection of tumour cells is most likely with progressing tumour development, which, apart from allowing a prognosis, also allows an evaluation of the therapeutic success.

This relation was also shown in a plurality of clinical studies, amongst others e.g. by Stathopoulou A. et al. (2002), “Molecular detection of cytokeratin-19-positive cells in the peripheral blood of patients with operable breast cancer: evaluation of their prognostic significance.” J. Clin. Oncol. 20(16): 3404-12.

Xenidis et al. (2003), “Peripheral blood circulating cytokeratin-19 mRNA-positive cells after the completion of adjuvant chemotherapy in patients with operable breast cancer” Ann. Oncol. 14(6): 849-55, Giatromanolaki et al. (2004), “Assessment of highly angiogenic and disseminated in the peripheral blood disease in breast cancer patients for resistance to adjuvant chemotherapy and early relapse.” Int. J. Cancer 108(4): 620-7 and Jotsuka T. et al. (2004), “Persistent evidence of circulating tumor cells detected by means of RT-PCR for CEA mRNA predicts early relapse: a prospective study in node-negative breast cancer.” Surgery 135(4): 419-26.

Thus, in the case of several forms of solid tumours, e.g. breast cancer, disseminated tumour cells in the bone mark (DTCs) as well as in the blood (CTCs) are an early marker for primary tumour development and possible metastasis as well as a negative survival prognosis for individuals suffering from these tumours.

A plurality of different methods for the determination of CTCs and DTCs are known (cf. Riethdorf et al., International Journal of Cancer, Vol. 123, 9^{th} Edition, p. 1991-2006 (November 2008)).

As stated above, it is known that CTCs and DTCs do not only occur with clinically evident, manifest tumours. CTCs have been observed, for example, in isolated cases of patients with non-detectable tumour diseases. Also in these cases, the CTCs are considered to be a surrogate marker for the existence of a minimal or developing tumour disease (Hirsch-Ginsberg (1998) Ann. Rev. Med. 49. 111-122).

From the prior art, it is known that tumour size correlates positively with the number of CTCs in the blood, imaging methods (such as CT/NMR/PET/sonography) for tumour diagnosis, however, have a resolving power (detection limit) which is 5 mm at best. At this stage, however, more than 500,000 tumour cells exist on site. Thus, smaller tumours cannot be detected with imaging methods.

However, CTCs can be already detected in the blood in the case of smallest tumours <5 mm, i.e. in early tumour stages and tumours that cannot yet be detected clinically with imaging methods (such as CT/NMR/PET/sonography).

For this reason, CTC diagnostics is used for early diagnosis of tumour diseases that cannot yet be detected with imaging methods; e.g. in cancer prevention in case of hereditary disposition for cancer (Detect early cancer in “healthy” people. The Kuhn Lab • 10550 North Torrey Pines Road • GAC-1200• La Jolla • Calif. • 92037; Zeidmann I: The fate of circulating tumor cells. I. Passage of cells through capillaries. Cancer Re 21 (1961) 38-39; and Hirsch-Ginsberg C. Annu Rev Med. 1998; 49:111-22. Detection of minimal residual disease: relevance for diagnosis and treatment of human malignancies).

Consequently, CTCs also provide information regarding the “seeding”, the dissemination of CTCs from tumours, and, thus, allow better information regarding the lymph node status (affected or unaffected) of cancer patients in comparison to imaging methods.

CTC diagnostics has many advantages over conventional tumour diagnostics since it allows a much more differentiated diagnosis.

In the past, the immediate therapeutic success during and subsequent to therapy could only be determined by means of tumour markers such as CEA, PSA, CA15-3, CA125, CA19-9, NSE, TPA and CYFRA21-1.

A reduction of tumour markers during or subsequent to therapy is attributed to therapeutic success. An increase in tumour markers during or subsequent to therapy does not allow differentiation between therapeutic success and failure (progression of the tumour disease) since, in general, the tumour marker increases in both cases. The introduction of CTC detection now allows a differentiated diagnosis.
Simultaneous increase in tumour markers and increase in CTCs represents therapeutic failure (progression of the tumour disease).
An increase in the tumour markers and a simultaneous reduction of CTCs represents therapeutic success: in this case, the increase in tumour markers correlates with an increased release of proteins from tumour cells upon their death due to therapy and, thus, fewer CTCs can be detected.
Thus, only the combination of both detection methods provide exact information with respect to the underlying processes.

### DESCRIPTION OF THE INVENTION

The present invention, in contrast, provides a pharmaceutical composition which directly aims at disseminated tumour cells in the blood as well as in the bone marrow as therapeutic target in order to counteract metastasis in the case of a manifest primary tumour, in particular solid tumours (distant metastasis) on the one hand. Since CTCs can be already be detected prior to the conventional detection of tumours in blood, on the other hand, early treatment is provided for patients with CTCs and without manifest primary tumour.

The present invention further provides a pharmaceutical composition for use in the early therapy of relapse or late therapy if, after surgical removal of the tumour or after radio- or chemotherapy, full remission is diagnosed (i.e. no detection of tumour or metastases) by means of imaging methods, despite CTCs still being present in the blood. A further subject matter of the invention comprises the therapy in the final stage of tumour diseases, i.e. with patients for whom the repertoire of conventional therapeutic possibilities has been exhausted without successful therapy (i.e. with patients without further therapeutic possibilities) for Ultima Ratio Therapy.

The present invention is based on the surprising findings that choline or pharmaceutically acceptable salts and analogues thereof have a direct effect on the CTCs, DTCs and pathologically modified leukocytes and their dysfunctional precursors in the blood and bone marrow.

Since they can be detected before metastases develop and in the case of small tumours even prior to the primary tumour, CTCs and DTCs can be considered a cancer entity of their own or have at least a cancerous property that is not necessarily, and not in a linear manner, associated with the primary tumour or the metastases. If CTCs can already be detected in early stages of a tumour disease, therapy can be carried out earlier with a higher therapeutic success to be expected which is initially independent from the primary tumour.

Thus the pharmaceutical composition according to the present invention allows the treatment of cancer diseases at early stages, at a stage when the cancer disease is not yet clinically manifest and cannot yet be detected by means of imaging methods such as CT, NMR, PET or sonography.

According to the present knowledge in classic medicine, this can also be considered early therapy.

Thus, it was surprisingly found that choline or pharmaceutically salts or analogues thereof can be used as pharmaceutical composition for use in early therapy of a tumour disease in patients without manifest primary tumour with CTCs, DTCs or dysfunctional leukocyte precursors being present in the blood or bone marrow.

Since often, even after full remission of a tumour, CTCs and DTCs are still present, the pharmaceutical composition of the invention can also be used for early therapy of a relapse or late therapy.

Moreover, it has been surprisingly found that choline or pharmaceutically acceptable salts and analogues thereof can be used as a pharmaceutical composition for use in tumour therapy for the reduction of metastasis risk and for the early therapy of metastases as well as in the therapy of leukaemias.

Tumour diseases according to the invention are in particular breast cancer, prostate cancer, pancreas cancer, gastroenteric cancer, skin cancer such as malignant melanoma, sarcoma such as histiocytoma and types of leukaemia.

Since CTCs and DTCs are even increased in final stage tumour patients after, according to common clinical criteria, unsuccessful therapy and are of pathologic and prognostic relevance, the pharmaceutical composition of the invention can also be used for the therapy (Ultima Ratio Therapy) of these patients.

In a first embodiment, the present invention relates to new pharmaceutical compositions, in particular for intravenous administration, containing choline or pharmaceutically acceptable salts and analogues thereof.

The present invention further relates to pharmaceutical compositions, in particular for intravenous administration, which comprise the above-mentioned compounds optionally together with vitamin C and/or L-arginine and/or vitamin D3 (cholecalciferol) or pharmaceutically acceptable salts and analogues thereof.

In the context of the present invention, “pharmaceutically acceptable salts” are understood to be in particular salts of choline which are formed with organic salts, such as e.g. tartaric acid, acidic acid, lactic acid, succinic acid, amygdalic acid, malic acid or citric acid; or with inorganic acids, such as hydrochloric acid, hydrobromic acid, sulfuric acid or nitiric acid.

In the context of the present invention, the term “analogues” is understood to be sphingomyelin, glycerophosphocholine, phosphatidylcholine, lecithin and phosphocholine as well as in particular esters of choline with C₁ to C₆ mono- and dicarboxic acids.

In the context of the invention, pharmaceutical compositions for intravenous administration are preferred, wherein the salts of choline are in particular selected from choline hydrogen tartrate and choline chloride and the also contained salt of L-arginine is preferably present as Larginine•HCl. Furthermore, the pharmaceutical composition of the invention can comprise further amino acids such as L-lysine, L-cysteine and taurine and vitamin D3 (cholecalciferol).

The present invention further relates to pharmaceutical compositions for intravenous administration which comprise the above-mentioned compounds optionally together with one or more pharmaceutically acceptable excipients, adjuvants and additives commonly used in the field.

Herein, excipients and adjuvants to be used according to the invention comprise one or more selected from aqua destillata, Ringer's lactate solution, hypertonic saline, further electrolytes, fat solutions comprising medium-chain triglycerides and omega-3 fat solutions.

In a preferred embodiment, fatty acids such as neutral fats and omega-3 fat solutions are infused in parallel and simultaneously to the administration of the pharmaceutical composition of the invention.

Herein, additives to be used according to the invention comprise one or more selected from vitamin B6, vitamin B12, folic acid and other amino acids, in particular L-lysine, L-cysteine and taurine as well as electrolytes such as magnesium, calcium, manganese and molybdenum.

In a further preferred embodiment of the present invention, the pharmaceutical compositions described above are used in tumour therapy for the reduction of metastasis risk and for the prophylaxis of metastases, respectively.

The pharmaceutical composition of the invention is preferably used in the context of a tumour therapy of mamma carcinomas (breast cancer), gastroenteric carcinomas, prostate cancer, pancreatic cancer, skin cancer such as malignant melanoma, sarcoma such as histiocytoma and types of leukaemia with the possibility to apply acute or maintenance therapy.

In general, the daily intravenous dose of choline for a patient weighing 80 kg is within a range which is equivalent to 0.5 g to 100 g choline hydrogen tartrate or in a range which is equivalent to 6 mg/kg to 1.4 mg/kg (body weight) choline hydrogen tartrate and preferably equivalent to 30 mg/kg to 1.25 g/kg choline hydrogen tartrate.

In case of maintenance therapy, the daily dose of choline for a patient weighing 80 kg is preferably within a range which is equivalent to 0.5 g to 10 g choline hydrogen tartrate or which is equivalent to 6 mg/kg to 125 mg/kg choline hydrogen tartrate. In case of acute therapy, the daily dose of choline, for a patient weighing 80 kg is preferably within a range equivalent to 2 g to 30 g choline hydrogen tartrate or equivalent to 0.03 g/kg to 1.25 g/kg and particularly preferred equivalent to 0.6 g/kg to 1.25 g/kg choline hydrogen tartrate.

Subsequently, the initial choline dose can be increased stepwise to maximum dose in accordance with the therapy results.

With the pharmaceutical composition being used according to the invention, the daily dose of vitamin C/L-arginine each is within a range of 1 g to 100 g, preferably in a range of 4.21 g to 22.5 g and particularly preferred within a range of 7.5 g to 22.5 g. The daily dose of vitamin D3 (cholecalciferaol) is within a range of 100,000 IU to 1,000,000 IU and preferably within a range of 200,000 IU to 500,000 IU

The pharmaceutical composition of the invention can further be administered in combination with further drugs, in particular conventional anti-tumour agents. This can take place either simultaneously or sequentially. In the context of the invention, it was shown that this results in a synergistic, at least additive or even potentiating effect. Notwithstanding the comparable cytosensitivity of these ingredients, they, however, differ significantly in the underlying mechanisms of action. Due to this fact, it is possible to considerably reduce the often serious side effects of established tumour therapies and, thus, to improve the acceptance of these established therapy forms significantly.

The combined application with chemotherapeutic agents, e.g. taxanes such as paclitaxel or docetaxel; humanized monoclonal antibodies such as bevacizumab or trastuzumab; anthracyclines or capecitabine or combinations thereof, is particularly preferred.

When the pharmaceutical compositions are used according to the invention in combination with already established tumour therapies, the dosage of the established tumour therapies corresponds to the recommended standard dosage of these therapies.

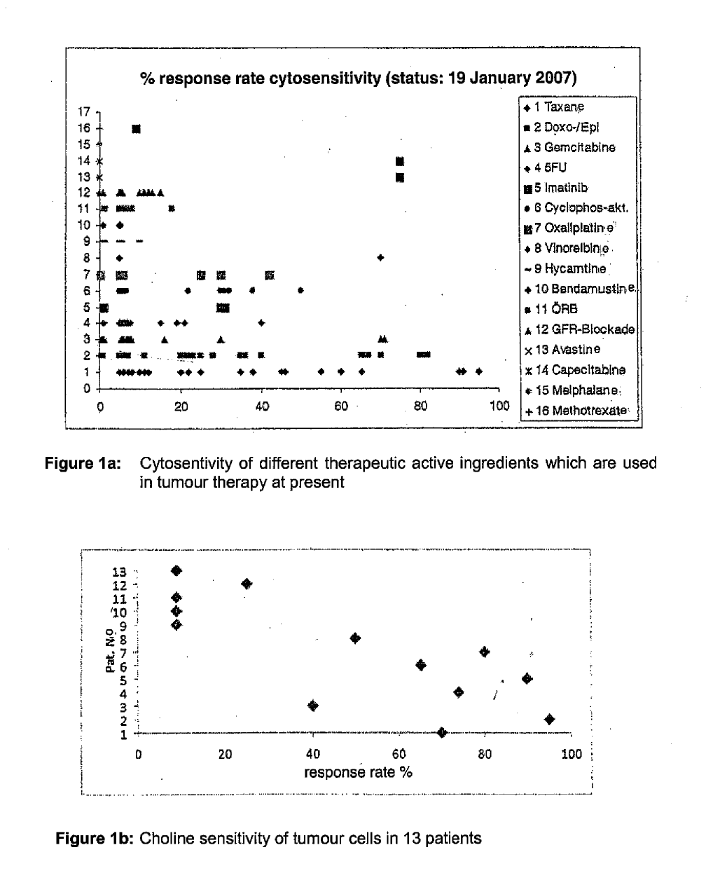

In the context of the invention, it was possible to show that with tumour diseases, in particular such as breast cancer, prostate cancer, gastroenteric cancer and pancreas cancer, the in vitro treatment of the tumour cells present in the patients' blood using the pharmaceutical composition of the invention allowed to achieve an anti-tumour effect (cytosensitivity) that corresponds in its degree to the effect of the anti-tumour agents which are at present used clinically (see FIG. 1a and 1b). This applies in particular to human medicine. This implies that the pharmaceutical composition of the invention has a direct effect on the CTCs and, thus, can be applied for early therapy without detection of a tumour, after full remission with detection of CTCs for early therapy of relapses or late therapy, for use in final stage tumour patients after unsuccessful therapy for Ultima Ratio Therapy as well as for the reduction of metastasis risk and for early therapy of metastases with a clinical manifest primary tumour.

The pharmaceutical compositions of the invention can be formulated/produced according to conventional methods and techniques known to the person skilled in the art; such as described e.g. in Remington's Pharmaceutical Sciences, 15^{th} edition, Mack Publishing Co., New Jersey (1991).

Apart from the preferred intravenous dosage forms, dosage forms for oral, parenteral (e.g. s.c., i.p., i.c., intrathecal) and local (e.g. topical, rectal, vaginal, buccal, application in the eye or by inhalation) are optionally also possible.

Thus, in addition to the i.v. administration, the pharmaceutical compositions of the invention can also be in form of tablets (also as enteric coated tablets or tablets with modified release of the active ingredient), capsules (hard or soft gelatine capsules), pills, granulates, suppositories, ovula, ointments, creams, gels, patches, TTS or as emulsions, suspensions, solutions or reconstitutable powders (also for parenteral application).

The following examples are evidence for the efficacy of the pharmaceutical composition of the invention.

### EXAMPLES

### 1. In Vitro Results—Effect on Disseminated Tumour Cells in the Blood (CTCs)

In 13 patients with detected tumour disease (breast cancer, colon cancer, prostate cancer, pancreas cancer), the direct effect of choline (cytosensitivity) on the tumour cells in the patients' blood was examined using the MAINTRAC® method. This method allows the detection of smallest amounts of tumour cells in blood and other body fluids (Pachmann K., Clement J. H., Schneider C.-P., Willen B., Camara O., Pachmann U., Hoeffken K., Standardized quantification of circulating peripheral tumour cells from lung and breast cancer. Clin. Chem. Lab-Med., 2005, 43; 617-627). It was possible to show that choline had an effect comparable to the one of conventional active ingredients (see FIG. 1a) in terms of the direct anti-tumoral effect of choline with these tumour cells showing a cytosensitivity to choline of up to 95% (see FIG. 1b). Thus, it was possible to detect for the first time in vitro the direct effect of choline on the circulating tumour cells (CTCs) in the blood.

### 2. In Vivo Results

In 9 patients with different tumour diseases (such as breast cancer, prostate cancer, pancreas cancer, gastric cancer, skin cancer, sarcoma and leukaemia), the direct in vivo effect on the patients' laboratory blood parameters (CTCs, leukocytes, dysfunctional leukocyte precursors) was examined comparing the values prior and subsequent to choline infusions in form of the pharmaceutical composition of the invention. The patients' blood was taken prior and subsequent to the infusion therapy, likewise, their vital parameters (RR, pulse, SO₂, ECG, temperature) were determined every quarter of an hour prior to, during and subsequent to the infusion therapy. Depending on the dose, the infusion therapy was applied for 3-5 hours. During and subsequent to the infusion therapy, which usually was very well tolerated, no patient showed relevant changes in the vital parameters. In some cases, side effects such as nausea and increased temperature were observed on the following day, in general only during few hours, depending on the dose.

However, after only few intravenous administrations, patients undergoing choline therapy regularly report a significant improvement of their health condition as well their physical resilience.

The values obtained are provided in Tables 1 and 2 below.

TABLE 1
In vivo effect of intravenous therapy with choline for 3 to 5 hours on the
circulating tumour cells (CTCs) in the patients' blood (MainTrac analysis).

| | | | | After − | After − |
|---|---|---|---|---|---|
| | | | | before | before |
| | | Before | After | Difference | Δ |
| Type of tumour | Parameters | therapy | therapy | (Δ) | % before |
| Mamma CA | MainTrac | 18.75 | 10.00 | −8.75 | −46.00% |
| Mamma CA | MainTrac | 5.25 | 2.00 | −3.25 | −61.00% |
| Mamma CA | MainTrac | 1.75 | 1.25 | −0.50 | −28.00% |
| Pancreas CA | MainTrac | 32.50 | 3.00 | −29.50 | −90.00% |
| Pancreas CA | MainTrac | 3.00 | 2.75 | −0.25 | −8.00% |
| Gastric CA | MainTrac | 14.00 | 1.75 | −12.25 | −87.00% |
| Prostate CA | MainTrac | 12.50 | 8.75 | −3.75 | −30.00% |
| Malignant | MainTrac | 3.75 | 0.00 | −3.75 | −100.00% |
| melanoma | | | | | |
| Malignant | MainTrac | 3.50 | 3.00 | −0.50 | −14.00% |
| melanoma | | | | | |
| Histiocytoma | MainTrac | 372.50 | 65.00 | −307.50 | −82.00% |
| Mean value | | 46.75 | 9.75 | −37.00 | −54.60% |

As illustrated above, after only one intravenous therapy with choline for 3 to 5 hours, the circulating tumour cells (CTCs) in the blood of patients suffering from diagnosed mamma cancer, prancreas cancer, gastric cancer, prostate cancer, malignant melanoma (skin cancer) and histiocytoma are reduced on average from 46.75 million tumour cells prior to the therapy to 9.75 million tumour cells subsequent to the therapy by −37 million tumour cells to −54.6% of the initial value prior to therapy. According to the technical literature, a reduction of the circulating tumour cells detected in the blood of these patients correlates with a better prognosis of these diseases and with a reduced risk of developing metastases.

TABLE 2
In vivo effect of an intravenous therapy with choline for 3 to 5 hours on
increased and in particular dysfunctional leukocyte precursors present
in the blood of leukaemia patients (laboratory diagnosis according
to the Guideline for Quality Assurance for Examination in Laboratory
Medicine of the German Medical Association¹)

| | | | | After − | After − |
|---|---|---|---|---|---|
| | | | | before | before |
| | Leukaemia | Before | After | Difference | Δ |
| Parameters | Evaluation | therapy | therapy | (Δ) | % before |
| Leukocytes | increased | 174.40 | 154.40 | −20.00 | −11.00% |
| Leukocytes | increased | 306.90 | 265.20 | −41.70 | −13.00% |
| Leukocytes | increased | 281.60 | 266.00 | −15.60 | −5.00% |
| Mean values | | 254.30 | 228.53 | −25.77 | −10.00% |

¹)Richtlinie der Bundesärztekammer zur Qualitätssicherung laboratoriumsmedizinischer Untersuchungen

As is known, leukaemias are characterised by highly increased formation of white blood cells (leukocytes) and, in particular, of their dysfunctional precursors. These leukaemia cells spread in the bone marrow, there they block normal haematopoiesis and, in general, their occurrence in peripheral blood is also highly increased. These dysfunctional precursors of leukocytes, like the CTCs in the case of tumour diseases, are eliminated by choline infusion therapy.

When the pharmaceutical compositions are used according to the invention, the detected circulating tumour cells (CTCs) in the blood of cancer patients as well as the increased and in particular dysfunctional precursors of leukocytes detected in the blood of leukaemia patients are acutely reduced, as shown in Tables 1 and 2, when compared immediately before and immediately after an intravenous therapy.

Even after several weeks, some of the acute changes in the patients' blood parameters, as shown in Tables 1 and 2, could still be detected to a significant degree. In the case of leukaemia and breast cancer, an acute choline infusion therapy according to the invention with maximum dose resulted in a sustained positive effect on these parameters over a period of 4 weeks.

TABLE 3
Change of the values during the therapy, prior/subsequent to therapy and 4 weeks later.
(Effect on the increased and in particular dysfunctional precursors of leukocytes
present in the blood of a leukaemia patient (laboratory diagnosis according to the
Guideline for Quality Assurance for Examination in Laboratory Medicine of the
German Medical Association²) and on the circulating tumour cells (CTCs) in the
blood of a patient suffering from breast cancer (MainTrac Analysis)).

| | | | | After − | After − | | After − |
|---|---|---|---|---|---|---|---|
| | | | | before | before | After 4 | before |
| | Sustainability | Before | After | Difference | Δ% | weeks | Δ% |
| Parameters | Evaluation | therapy | therapy | (Δ) | before | value | before |
| Leukocytes | increased | 306.90 | 265.00 | −41.90 | −13.00% | 281.60 | −8.00% |
| MainTrac | increased | 18.75 | 10.00 | −8.75 | −46.00% | 5.25 | −72.00% |
| (CTCs) | | | | | | | |
| Mean values | | | | | −30.00% | | −40.00% |

²Richtlinie der Bundesärztekammer zur Qualitätssicherung laboratoriumsmedizinischer Untersuchungen

As illustrated in Table 3 above, after only one intravenous therapy with choline for 3 to 5 hours, the leukocytes measured in the blood of a leukaemia patient before and after therapy were reduced by −13% and, 4 weeks later, there were still reduced by significant −8% compared to the initial value. In a patient suffering from breast cancer, the circulating tumour cells (CTCs) in the patient's blood, detected by MainTrac analysis before and after therapy, were reduced by −46% of the initial value from 18.75 million tumour cells before therapy to 10.00 million tumour cells after therapy. After another 4 weeks without therapy, this value was further reduced to 5.25 million tumour cells and, thus, to −72.00% of the initial value before therapy.

According to the technical literature, a reduction of the circulating tumour cells in the blood of this patient and dysfunctional precursors of leukocytes detected correlates with a better prognosis of this disease as well as with a reduced risk of developing metastasis.

1. Pharmaceutical composition containing choline or pharmaceutically acceptable salts and analogues thereof for the use for elimination or reduction of CTCs, DTCs or dysfunctional leukocyte precursors in the blood and bone marrow.

2. Pharmaceutical composition for the use according to claim 1, for use in tumour therapy for the reduction of metastasis risk and for the prophylaxis of metastases (distant metastasis) with clinically manifest primary tumour.

3. Pharmaceutical composition for the use according to claim 1, for use prior to occurrence of a clinically manifest primary tumour for early treatment.

4. Pharmaceutical composition for the use according to claim 1, for use after full remission for late therapy.

5. Pharmaceutical composition for the use according to claim 1, for use in final stage tumour patients after, according to common criteria, unsuccessful therapy for Ultima Ratio Therapy.

6. Pharmaceutical composition for the use according to claim 1, for use in therapy of leukaemias.

7. Pharmaceutical composition according to claim 1, further containing vitamin C and/or vitamin D3 (cholecalciferol).

8. Pharmaceutical composition according to claim 1, further containing L-arginine or pharmaceutically acceptable salts thereof.

9. Pharmaceutical composition according to claim 1, wherein the pharmaceutically acceptable salt of the choline is selected from choline hydrogen tartrate and choline chloride.

10. Pharmaceutical composition according to claim 1, wherein the pharmaceutically acceptable salt of the L-arginine is L-arginine.HCl.

11. Pharmaceutical composition according to claim 1, wherein the tumour disease is selected from breast cancer, colon cancer, gastric cancer, prostate cancer, pancreas cancer, skin cancer, sarcoma and leukaemia.

12. The pharmaceutical composition for the use according to claim 1, wherein the daily dose is within a range which is equivalent to 0.5 to 100 g choline hydrogen tartrate or equivalent to 6 mg/kg to 1.4 g/kg choline hydrogen tartrate and preferably equivalent to 30 mg/kg to 1.25 g/kg choline hydrogen tartrate.

13. The pharmaceutical composition for the use according to claim 1, wherein the tumour therapy is selected from maintenance therapy and acute therapy.

14. The pharmaceutical composition for the use according to claim 13, wherein, in the case of maintenance therapy, the daily choline dose is within a range which is equivalent to 0.5 g to 10 g choline hydrogen tartrate or equivalent to 6 mg/kg to 125 mg/kg choline hydrogen tartrate.

15. The pharmaceutical composition for the use according to claim 13, wherein, in the case of acute therapy, the daily choline dose is within a range which is equivalent to 2 g to 30 g choline hydrogen tartrate or equivalent to 0.03 g/kg to 1.25 g/kg and particularly preferred equivalent to 0.6 g/kg to 1.25 g/kg choline hydrogen tartrate.

16. The pharmaceutical composition for the use according to claim 1, wherein each, the daily dose of vitamin C and the daily dose of L-arginine is within a range of 1 g to 100 g, preferably within a range of 4.21 g to 22.5 g and particularly preferred within a range of 7.5 g to 22.5 g.

17. The pharmaceutical composition for the use according to claim 1, wherein the daily dose of vitamin D3 is within a range of 100,000 IU to 1,000,000 IU and preferably within a range of 200,000 IU to 500,000 IU.

18. The pharmaceutical composition for the use according to claim 1 for intravenous administration.

19. The pharmaceutical composition for the use according to claim 1 for use in combination with one or more anti-tumour agent(s).

20. The pharmaceutical composition for the use of claim 19 for simultaneous or sequential application.

21. The pharmaceutical composition for the use according to claim 19, wherein the anti-tumour agent is selected from taxanes such as paclitaxel or docetaxel; humanized monoclonal antibodies such as bevacizumab or trastuzumab; anthracyclines or capecitabine or combinations thereof.
